(19) United States
(12) Patent Application Publication
(10) Pub. No.: US 2024/0238553 A1
(43) Pub. Date: Jul. 18, 2024

(54) EXPIRATION VALVE ASSEMBLY AND BREATHING GAS LINE ASSEMBLY COMPRISING SUCH AN ASSEMBLY

(71) Applicant: Hamilton Medical AG, Bonaduz (CH)

(72) Inventors: Kerstin Lipinski, Trin (CH); Kim Haltiner, Chur (CH)

(21) Appl. No.: 18/576,271

(22) PCT Filed: Jul. 1, 2022
(86) PCT No.: PCT/EP2022/068277

§ 371 Date: Jan. 3, 2024

(30) Foreign Application Priority Data
Jul. 6, 2021 (DE) 10 2021 117 375.2

(51) Int. Cl. A61M 16/20; A61M 16/00; A61M 16/08

(52) U.S. Cl. CPC A61M 16/208; A61M 16/0003; A61M 16/0833; A61M 16/0875; A61M 2016/0027; A61M 2016/0036

(57) ABSTRACT

An expiration valve assembly for a ventilator, including an expiration channel which has, at one end, an expiration outlet and an inlet connection part that is designed to connect to an expiration breathing gas line and which, at its other end, has an expiration outlet, the expiration channel having an expiration valve; an inspiration channel which has, at one end, an inspiration inlet and an inlet connecting part that is designed to connect to a breathing gas source supplying inspiratory breathing gas, and which, at its other end, has an inspiration outlet and an outlet connecting part that is designed to connect to an inspiration breathing gas line; a control channel which branches off from the inspiration channel at a branch location and leads to the expiration valve in such a way that the expiration valve can be loaded by inspiratory breathing gas into a blocking position which blocks the flow of expiratory breathing gas; a one-way valve being arranged in the inspiration channel and permits a flow of inspiratory breathing gas in the inspiration direction from the inspiration inlet to the inspiration outlet and prevents a flow of breathing gas in the opposite direction.

This application claims priority in PCT application PCT/EP2022/068277 filed Jul. 1, 2022, which claims priority in German Patent Application DE 10 2021 117 375.2 filed on Jul. 6, 2021, which are incorporated by reference herein.

The present invention concerns an expiration valve assembly for a respiratory device for artificial ventilation of a patient, comprising
An expiration duct which at one end exhibits an expiration inlet for introducing an expiratory breathing gas flow into the expiration duct and an inlet linkage formation which is configured for linking to an expiration breathing gas line leading to the patient, and which at its other end exhibits an expiration outlet for discharging expiratory breathing gas, where the expiration duct exhibits an expiration valve which through an expiratory breathing gas flow in the expiration direction from the expiration inlet to the expiration outlet is moveable into a feed-through position which lets the expiratory breathing gas flow through,
An inspiration duct which exhibits at one end an inspiration inlet for introducing an inspiratory breathing gas flow into the inspiration duct and an inlet connector formation which is configured for connecting with a breathing gas source supplying inspiratory breathing gas, and which at its other end exhibits an inspiration outlet for letting out inspiratory breathing gas and an outlet connector formation which is configured for connecting with an inspiration breathing gas line leading to the patient,
A control duct which branches off from the inspiration duct at a branching point and leads to the expiration valve in such a way that the expiration valve can be strained by inspiratory breathing gas into a blocking position which blocks the expiratory breathing gas flow.

### BACKGROUND OF THE INVENTION

Such an expiration valve assembly configured to conduct both an expiratory and an inspiratory breathing gas flow is known for example from WO 2021/018902 A1.

The expiration valve assembly known from WO 2021/018902 A1 is part of a breathing gas line assembly, with a hose as a line component connected to the expiration valve assembly. This hose is configured as a bi-lumen hose in such a way that within its outermost hose sleeve it exhibits both the expiration breathing gas line and the inspiration breathing gas line. To its distal longitudinal end, that is, the one further from the patient during operation, there is connected the known bi-lumen hose with the expiration valve assembly. At its proximal longitudinal end, that is, the one nearer to the patient during operation, there is arranged at the hose a Y-connecting element with a check valve assembly. The Y-connecting element leads together, between its distal longitudinal end with two arms and its proximal longitudinal end with one arm, the distally connected breathing gas lines: expiration and inspiration breathing gas line, in a common bidirectional breathing gas line.

The check valve assembly serves, in the region of the respective currently inactive breathing gas line out of inspiration and expiration breathing gas line lying distally to the check valve assembly, to decouple existing breathing gas columns from the respective other, active breathing gas line. Thereby, breathing gas should flow only through the active breathing gas line desired in respective ventilation phase.

However, by accommodating the check valve assembly, the Y-connecting element requires a relatively large installation space and becomes cumbersome. This is disadvantageous precisely for the Y-connecting element which is always arranged near the patient.

The known expiration valve arranged in the expiration duct is responsible for blocking of the expiration duct, in that when during an inspiration phase inspiratory breathing gas flows in the inspiration direction through the inspiration duct, the expiration valve is strained by branched-off inspiratory breathing gas into the blocking position. Under spontaneous inspiration of the patient, the underpressure created by the patient during the spontaneous inspiration ensures a straining of the expiration valve into the blocking position. In each of the aforementioned cases, there exists during an inspiration phase a pressure drop at the expiration valve between its in the expiration direction upstream side and its downstream side, which strains the expiration valve into the blocking position.

In the breathing gas line assembly known from WO 2021/018902 A1, a check valve in the Y-connecting element ensures similar blocking of the inspiration breathing gas line during expiration. It prevents breathing gas flow in the inspiration breathing gas line in the expiration direction which is opposite to the inspiration direction.

As a further background to the state of the art, reference is made additionally to EP 2 663 354 B1.

### SUMMARY OF THE INVENTION

It is the task of the present invention to propose a technical approach which makes it possible to block the temporarily inactive breathing gas line in the respective ventilation phase out of inspiration and expiration for a throughflow in the direction which is wrong for the respectively inactive breathing gas line, without having to arrange cumbersome technical medical components permanently near the patient.

The present invention solves this task by having arranged in the inspiration duct of an expiration valve assembly of the kind mentioned at the beginning a one-way valve which permits inspiratory breathing gas flow in the inspiration direction from the inspiration inlet to the inspiration outlet and prevents breathing gas flow in the opposite direction.

In an advantageous manner, the expiration valve assembly which is always arranged remotely from the patient on the distal side of a ventilation hose or the like, thus provides during an inspiration a blocking of the expiration breathing gas line and during an expiration a blocking of the inspiration breathing gas line. The one-way valve necessary to this end can thus be arranged on the one hand in a relatively compact assembly, and on the other at a distance from the ventilated patient. Even if the expiration valve assembly, through the arrangement of the one-way valve inside it, were to increase in construction volume against the expiration valve assembly known from the state of the art, this would happen at such a great distance from the ventilated patient that the latter would not be restricted by this.

The one-way valve is preferably a simple check valve with a valve seat and a valve body, which in its closed position which closes the inspiration breathing gas line physically abuts against the valve seat and which is removeable from the valve seat to enable flow through the inspiration breathing gas line. In order to ensure that the inspiration breathing gas line can have throughflow only in the inspiration direction, the valve body can be raised from the valve seat at its fitting site in the expiration valve assembly only along the local inspiration direction. Thereby, the one-way valve can be displaced by the inspiratory breathing gas flow itself into its open position which is raised from the valve seat and consequently is throughflow-permitting, whereas breathing gas flow in the opposite direction strains the valve body back to its valve seat and thus into the closed position.

The valve body can be pre-tensioned into the closed position by an elastic force, for example through a pre-tensioning means such as for instance a pre-tensioning spring or an elastic polymer pre-tensioning element. An advantageous simplification by way of having the smallest number of components can be obtained by the valve body being its own pre-tensioning element. Preferably, therefore, the valve body is deformable by the inspiratory breathing gas flow between a closing shape and an opening shape. In the closing shape, the valve body abuts against the valve seat and closes the inspiration duct. In the opening shape, the valve body is at least section-wise raised from the valve seat and allows flow through the inspiration duct in the inspiration direction. An additional pre-tensioning component can thereby be dispensed with.

According to the present invention, the valve body can be configured as a pivotable flap, where in order to realize the aforementioned deformability between closing shape and opening shape, a hinge which permits the pivoting movement of the flap-like configured valve body is preferably configured as a film hinge.

Alternatively, and because of the possibility of a symmetric throughflow preferably, the valve body can be configured as a disc which is attached in a central disc region lying distantly from its disc edge such that the disc edge can move relative to the attached central disc region through deformation of the disc transversely to the disc area. The valve seat can then be formed by an annular area against which in the closed position of the one-way valve a disc edge region lying radially outside the central disc region abuts, and from which the disc edge region can be raised through deformation of the disc-shaped valve body in the opening shape.

In order to attach the valve body, it can exhibit in the central disc region an aperture penetrating through it through which an attachment component penetrates. The attachment component preferably exhibits on both sides of the aperture being penetrated a larger cross-section than the penetrated aperture, such that the valve body can be very effectively positively attached by the attachment component.

The valve seat and the attachment component of the valve body are preferably configured as an integral component. The one-way valve is preferably insertable into the inspiration duct as a preassembled assembly, comprising the basic component with the valve seat and the attachment component. Preferably the component, especially preferably as an integral component, with valve seat and attachment component fixable in the inspiration duct after insertion into it, either through gluing, welding such as for example ultrasound or friction welding, or simply frictionally engaged through friction between an external surface of a component wall facing towards the internal surface of the inspiration duct wall and the internal surface. For facilitated insertion, the attachment component can be configured rotation-symmetrically at least with its fixing section which is fixable at the inspiration duct or with shape sections which repeat along the circumferential direction, if the section of the inspiration duct into which the basic component with the one-way valve is inserted is likewise configured rotation-symmetrically. Then advantageously the orientation of the basic component with respect to the insertion axis is unimportant during assembly.

To facilitate thermal fixing through melting of boundary surfaces which lie against one another, such as for instance in welding, at least the fixing section of the basic component and the section of the inspiration duct accommodating it are fabricated from compatible thermoplastic synthetics at least at the surfaces which lie against one another and touch one another. To simplify fabrication, preferably the entire basic component with valve seat and attachment component and/or the entire inspiration duct is formed by compatible thermoplastic materials.

The valve body, in particular the disc-shaped valve body, can be formed from a thermoplastic elastomer or generally from an elastomer, such as for instance silicone. The material of the valve body does not have to be compatible with the material of the valve seat and/or of the attachment component and/or with the material forming the inspiration duct wall. Indeed, preferably it is not, in order to avoid undesirable random bonds between the valve body and the valve seat and/or the inspiration duct wall.

The inspiration direction and the expiration direction are functionally opposite directions, which however does not mean that at every point in the expiration valve assembly the inspiratory breathing gas stream and the expiratory breathing gas stream flow in respectively opposite flow directions. The expiration duct and the inspiration duct can proceed in parallel section-wise, but usually do not proceed in parallel along their entire common extension since for example the expiration outlet, which preferably lies in the arrangement region of the expiration valve, opens into the external environment of the expiration valve assembly whereas the inspiration inlet is connected to a breathing gas source, such as for example to a breathing gas reservoir under pressure and/or to a fan or the like. If the expiration duct and the inspiration duct extend locally in parallel at or in the expiration valve assembly, in this parallel region the expiratory and the inspiratory breathing gas flows do in fact normally flow in opposite flow directions.

The inlet linkage formation, the inlet connector formation, and the outlet connector formation can exhibit arbitrary shapes which make possible the linking of a further line or a further duct. The aforementioned formations can each be part of a pneumatic quick coupling or can simply be formed only by either a duct fitting or a duct socket. Experts normally know how breathing gas lines should be connected to an inlet linkage formation and an outlet connector formation. Likewise it is known to experts how an inlet connector formation can be connected fluidically effectively with a breathing gas reservoir, i.e. for instance a container with breathing gas stored in it under pressure or a ventilator with a fan.

In order to ensure that the expiration valve, irrespective of the functionality of the one-way valve, can be strained by inspiratory breathing gas into the blocking position, preferably the branching point lies upstream of the one-way valve in the inspiration direction.

The expiration valve is preferably configured as a membrane valve which is known per se, with a membrane which is deflectable orthogonally to its main extension surface as a valve body and with an end-face longitudinal end of a section of the expiration duct as an annular valve seat against which the valve body abuts in the blocking position.

The expiration valve preferably exhibits in a manner which is known per se a radially inner inlet-side expiration duct section exhibiting the expiration inlet with valve seat and an outlet-side expiration duct section lying radially outside the inlet-side expiration duct section and exhibiting the expiration outlet. Only in the feed-through position, i.e. with valve body raised from the valve seat, can expiratory breathing gas overflow from the inlet-side into the outlet-side expiration duct section. Due to the relatively large membrane area, on the side of the preferred membrane valve body facing away from the valve seat there can be configured a chamber which communicates with the control duct, such that inspiratory breathing gas can flow through the control duct into the chamber and there strain the membrane valve body towards the valve seat, i.e. into its blocking position.

The membrane valve body preferably has an inspiratory membrane area facing towards the chamber which is wettable by inspiratory breathing gas. On the opposite side facing towards the expiration inlet, the membrane valve body preferably exhibits an expiratory membrane area which can have inflow of expiratory breathing gas from the inlet-side expiration duct section. When regarding the expiration valve in the closed state as a reference state, the expiratory membrane area is situated inside the valve seat. Preferably, the inspiratory membrane area of the membrane valve body which is wettable by inspiratory breathing gas is larger than the expiratory membrane area which can have inflow of expiratory breathing gas, in order to be able to hold the expiration valve securely closed by inspiratory breathing gas during an inspiration process. An advantageous area ratio of inspiratory to expiratory membrane area lies in the range from 1.5 to 2. More preferably, the area ratio lies in the range from 1.7 to 1.9. Especially preferably, the area ratio equals 1.8.

In principle, the inspiration duct and the expiration duct can exhibit arbitrary courses between their respective inlet and their respective outlet. In order to avoid unnecessary vortices and flow resistances, however, it is preferable that an inspiration inlet section of the inspiration duct which lies nearer to the inspiration inlet than to the inspiration outlet proceeds along an inspiration inlet axis. The inspiration inlet section preferably exhibits the inspiration inlet and extends starting out from it.

For clarification let it be noted that in the present application the term ‘axis’ denotes a straight path.

In WO 2021/018902 A1, the valve movement path along which the valve body of the expiration valve can be raised from its valve seat proceeds orthogonally to the inspiration inlet axis. The known valve movement path furthermore proceeds in parallel to an inspiration outlet section which lies nearer to the inspiration outlet than to the inspiration inlet. The known inspiration outlet section proceeds along an inspiration outlet axis. Moreover, the known valve movement path proceeds in parallel to an expiration inlet section which lies nearer to the expiration inlet than to the expiration outlet and proceeds along an expiration inlet axis. With these kinematics of the valve body, in some operational situations the gravitational force can have an adverse effect on the expiration valve or at least no advantageous effect on it.

In principle, the valve body of the expiration valve is pre-tensioned in the blocking position, normally through the material elasticity of the preferred membrane valve body. In order to be able to ensure additionally in most operational situations that the gravitational force strains the expiration valve into the blocking position and thus increases the functional reliability of the expiration valve, in the expiration valve assembly discussed here it is preferably provided that a valve movement path along which a valve body, in particular the aforementioned membrane valve body, of the expiration valve can be raised from a valve seat, in particular the aforementioned valve seat, of the expiration valve from its blocking position and can be made to approach the valve seat, is tilted relative to the inspiration inlet axis by a setting angle in the range from 10° to 45°, preferably in the range from 15° to 35°. Preferably the expiration valve assembly discussed here is deployed in an emergency ventilator which is carried in patient or accident emergency vehicles such as emergency ambulances, rescue helicopters etc. The orientation of the expiration valve assembly in emergency deployment is accordingly unforeseeable. The stated position of the expiration valve with respect to the valve movement path has shown that in most operational situations, the expiration valve assembly comes to be deployed oriented in such a way that at least one part of the gravitational force acting on the valve body strains the latter into the blocking position.

An advantageously compact expiration valve assembly, with further lines connected to the linkage and connector formations of the expiration valve assembly in the application situation, can be obtained by having an expiration inlet section of the expiration duct which lies nearer to the expiration inlet than to the expiration outlet proceeding along an expiration inlet axis. Preferably, the valve movement path is tilted about a parallel to the expiration inlet axis with respect to the inspiration inlet axis.

The expiration inlet axis and the inspiration inlet axis are preferably oriented orthogonally to one another. The two inlet axes, imagined as penetrating through the respective duct section centrally, do not have to intersect but can pass by one another at a distance.

The valve seat is preferably planar or has a valve seat surface lying in a plane, as the case may be. If the valve seat surface exhibits an extension along the valve movement path, for instance because it is conical or negative-conical, the aforementioned plane of the valve seat surface should not be understood as a mathematical plane but rather as a technical plane with a small extension along the valve movement path.

When the valve movement path is tilted to the inspiration inlet axis in accordance with the above elucidation, preferably the valve seat or the extension plane of the planar valve seat surface is also tilted relative to a plane orthogonal to the inspiration inlet axis. In this case, the valve seat, including a theoretically possible non-planar valve seat, exhibits a proximity section approaching and tilted towards the inspiration inlet section and a distanced section lying further away and tilted away from the inspiration inlet section. In order to be able to keep the control duct as short as possible and thereby as loss-free as possible, the control duct preferably runs nearer to the proximity section than to the distanced section from the branching point to the expiration valve.

The control duct can at least section-wise be configured as a duct component or duct component section respectively arranged spatially at a distance from the inspiration duct and/or from the expiration duct. Thereby, in contrast to the integrated construction of the control duct with at least one duct out of the inspiration duct and expiration duct, each of these ducts can be provided with the smallest possible cross-section and thus the expiration valve assembly with the smallest possible construction volume.

Preferably, in the present expiration valve assembly too it is considered to let any necessary signaling lines run at least section-wise through the expiration valve assembly, in particular through at least one duct out of the inspiration duct and expiration duct. So that such an at least one signaling line led in a duct can be securely led out of the latter and connected with its communicating device, according to a preferred further development of the present invention there can be configured in a region between the inspiration inlet section and the expiration inlet section and/or in a region between the inspiration inlet section and an inspiration outlet section lying nearer to the inspiration outlet than to the inspiration inlet at least one feed-through aperture. This at least one feed-through aperture penetrates through a duct wall which bounds the inspiration duct and/or the expiration duct.

The at least one feed-through aperture can be simply penetrated through by the at least one signaling line. Alternatively, a signaling line can end physically on the inside of the duct wall facing towards the respective duct. In order to position the longitudinal end of the signaling line securely, there can be arranged on the inside of the duct wall an accommodating formation, for instance a plug-in fitting, an insertion connector, or a ring of leaf springs protruding in the same direction from the circumferential edge region of the feed-through aperture, onto which or into which as the case may be the longitudinal end of the signaling line can be plugged, for producing a positive-fit accommodating engagement with the longitudinal end of the signaling line.

Likewise there can be arranged on the outside of the relevant duct a further accommodating formation which in an analogous manner is configured for positioning a longitudinal end of a further signaling line which continues the signaling line outside the duct leading the signaling line. The further accommodating formation too, can for example be a plug-in fitting, an insertion connector, or a ring of leaf springs protruding in the same direction from the circumferential edge region of the flow-through aperture. The at least one accommodating formation can be configured at a separate feedthrough component which can be inserted and fixed in a flow-through aperture,

When several flow-through apertures are configured, the flow-through apertures are preferably configured uniformly, likewise any possible plurality of feedthrough components.

In order to avoid the at least one signaling line having to be unnecessarily curved, where at all possible the at least one feed-through aperture is so arranged that the signaling line section or the further signaling line linking to the feed-through aperture do not have to be led around duct components. This can be achieved by the at least one feed-through aperture being located in the expiration direction downstream of a reference plane which is oriented orthogonally to the expiration inlet axis and subdivides an aperture surface bordered by the valve seat into equal area parts.

Furthermore, unnecessary curvature of the signaling line can be avoided by the at least one feed-through aperture and/or a feedthrough component that is possibly inserted in the feed-through aperture, proceeding along a feed-through axis which is parallel to the expiration inlet axis and/or to an inspiration outlet axis along which the inspiration outlet section extends.

It should be taken into consideration here that the signaling line normally proceeds from a sensor, in particular flow sensor, located nearer to the patient than the expiration valve assembly, to the expiration valve assembly. The signaling line can be an electrical signaling line which transmits electrical signals. Then normally its curvature is non-critical. Especially in the often-used proximal differential pressure flow sensors, two signaling lines are provided of which each as a hollow pressure line transmits a breathing gas pressure upstream of a flow resistance in the flow sensor and a breathing gas pressure downstream of the flow resistance. Avoiding curvatures and kinks is helpful so to achieve the most precise pressure transmission possible.

In principle it is possible to configure the expiration inlet section and the inspiration outlet section, which both functionally face towards the patient to be ventilated, spatially separated from one another. It is precisely in emergency medicine, however, that the quickest possible set-up of a ventilator and thereby also of the expiration valve assembly is a decisive advantage. Therefore it is preferably provided that the expiration inlet section and the inspiration outlet section are configured in a common multi-lumen end section. Accordingly, the inlet linkage formation and the outlet connector formation can be advantageously configured as a common, preferably single-piece, linkage connector formation. To these a multi-lumen hose can be connected with a single connecting procedure in such a way that at the same time and as far as possible with only one operation, both the expiration breathing gas line is connected to the expiration duct and the inspiration breathing gas line is connected to the inspiration duct.

The expiration valve assembly can be made as compact as possible by the inspiration inlet section, the expiration inlet section, and the inspiration outlet section being realized through an integral duct component. The integral duct component is preferably produced in the synthetic injection molding process. The integral duct component exhibits, preferably likewise configured at it integrally, the inlet linkage formation, the inlet connector formation, and the outlet connector formation, preferably each as a connector fitting and/or connector socket.

The present invention further concerns a breathing gas line assembly, comprising an expiration valve assembly, as described above and further developed, an expiration breathing gas line, an inspiration breathing gas line, and a flow sensor.

The expiration breathing gas line preferably exhibits at its distal longitudinal end a distal expiratory coupling formation which is configured to establish a connection which conducts an expiratory breathing gas flow with the inlet linkage formation of the expiration duct of the expiration valve assembly. Likewise, the expiration breathing gas line exhibits at its proximal longitudinal end a proximal expiratory coupling formation which is configured to establish a connection which conducts an expiratory breathing gas flow with an expiratory output linkage formation at the distal longitudinal end of the flow sensor. Furthermore, the inspiration breathing gas line exhibits at its distal longitudinal end a distal inspiratory coupling formation which is configured to establish a connection which conducts an inspiratory breathing gas flow with the outlet connector formation of the inspiration duct of the expiration valve assembly. Finally, the inspiration breathing gas line exhibits at its proximal longitudinal end a proximal inspiratory coupling formation which is configured to establish a connection which conducts an inspiratory breathing gas flow with an inspiratory input linkage formation at the distal longitudinal end of the flow sensor.

To avoid an unnecessarily large number of different lines or hoses in which rescue personnel or medical personnel could become entangled during deployment, preferably in at least one breathing gas line out of the expiration and inspiration breathing gas line there is accommodated at least one signaling line which is configured to transmit information acquired by the flow sensor from the proximal to the distal longitudinal end of the at least one breathing gas line. This is the at least one signaling line already mentioned above.

In such a breathing gas line assembly, the one-way valve described above in the inspiration duct of the expiration valve assembly is preferably the only one-way valve in the inspiratory breathing gas flow from the inspiration inlet to the proximal end of the flow sensor. A further valve arrangement in the inspiration duct and/or in the entire inspiration breathing gas line respectively is nit functionally necessary.

Likewise, the expiration valve is preferably the only valve arrangement in the expiration duct or especially preferably in the entire expiration breathing gas line.

As already set forth above, at least one of the at least one signaling line can penetrate through at least one of the at least one feed-through aperture or end in an accommodating engagement of an accommodating formation for accommodating the distal end of the at least one signaling line. Possible accommodating formations are already mentioned above.

For further avoidance of an unnecessarily large number of lines, in particular hoses, which increases the risk that personnel active in the operational region of the breathing gas line assembly becomes entangled and endangers or even terminates a ventilation of a patient, the expiration breathing gas line and the inspiration breathing gas line are preferably configured at a common multi-lumen line component. Indeed, the multi-lumen line component, for instance as a multi-lumen breathing gas hose, can also be connected to formations spatially separated from one another out of inlet linkage formation and outlet connector formation. Preferably, however, the multi-lumen line component is connected to the aforementioned multi-lumen end section of the expiration valve assembly.

Although the multi-lumen line component can, as a bi-lumen line component exhibit two coaxial, preferably concentric, hoses, in order to provide approximately equal cross-sectional areas in the inspiration as in the expiration breathing gas line with approximately equal wall area per distance unit it is preferable if the expiration and inspiration breathing gas lines are segregated from one another by a septum in the line component proceeding along the line component and along its internal diameter. The line component is preferably a hose.

In order to burden the two breathing gas lines out of expiration and inspiration breathing gas line as uniformly as possible by the accommodation of signaling lines, equally many signaling lines are preferably accommodated in the expiration lumen and in the inspiration lumen, preferably exactly one each.

At its proximal longitudinal end, the breathing gas line assembly, in particular the line component, especially preferably the multi-lumen line component, can exhibit a proximal coupling component with which the line component, in particular the multi-lumen line component, is connected. The coupling component preferably exhibits at its distal longitudinal end an expiration connector formation for establishing a connection which conducts an expiratory breathing gas flow with the expiration breathing gas line, in particular with the expiration lumen of the multi-lumen line component, and an inspiration connector formation for establishing a connection which conducts an inspiratory breathing gas flow with the inspiration breathing gas line, in particular with the inspiration lumen of the multi-lumen line component. The coupling component preferably exhibits at its proximal longitudinal end a coupling formation which is both the proximal expiratory coupling formation and the proximal inspiratory coupling formation.

These and other objects, aspects, features and advantages of the invention will become apparent to those skilled in the art upon a reading of the Detailed Description of the invention set forth below taken together with the drawings which will be described in the next section.

### BRIEF DESCRIPTION OF THE DRAWINGS

The invention may take physical form in certain parts and arrangement of parts, a preferred embodiment of which will be described in detail and illustrated in the accompanying drawings which forms a part hereof and wherein:

FIG. 1A An exploded side view of a distal end an embodiment according to the invention of a breathing gas line assembly with an embodiment according to the invention of an expiration valve assembly,

FIG. 1B An exploded side view of a proximal end of the breathing gas line assembly of FIG. 1A,

FIG. 2A A side view of the distal end of the breathing gas line assembly of FIG. 1A in the assembled state when viewed along of the arrow IIA in FIGS. 3A and 4,

FIG. 2B A side view of the proximal end of the breathing gas line assembly of FIG. 1B in the assembled state when viewed along of the arrow IIB in FIG. 3B,

FIG. 3A A bottom view of the proximal end of the breathing gas line assembly of FIG. 2A along of the arrow IIIA in FIGS. 2A and 4,

FIG. 3B A bottom view of the distal end of the breathing gas line assembly of FIG. 2B along of the arrow IIIB in FIG. 2B,

FIG. 4 An elevation view of the breathing gas line assembly when viewed along of the arrow IV in FIGS. 2B and 3B,

FIG. 5A longitudinal section of a one-piece duct component of the expiration valve assembly of FIG. 4 along the sectional plane V-V of FIG. 4.

### DESCRIPTION OF PREFERRED EMBODIMENTS

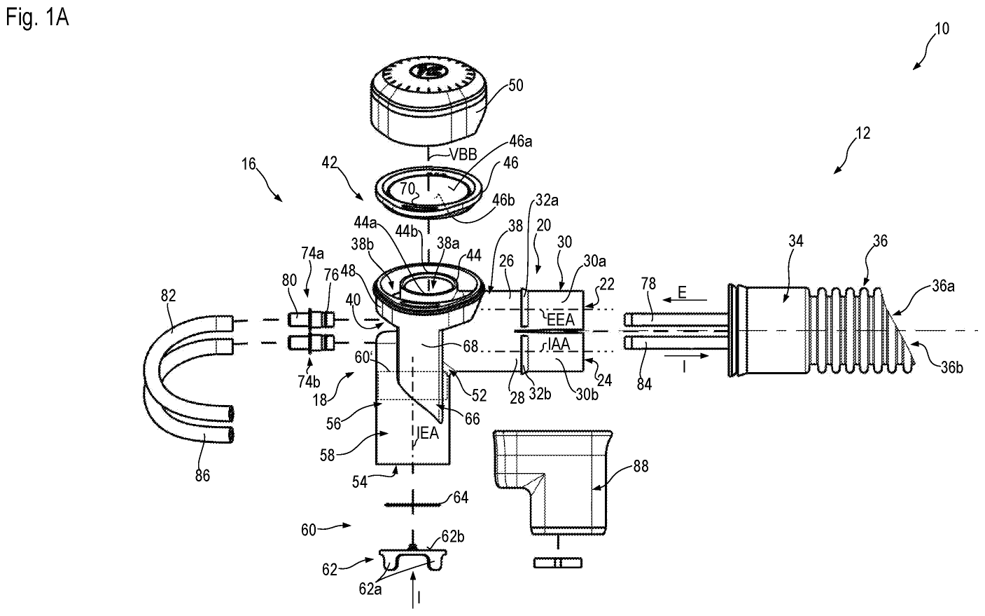

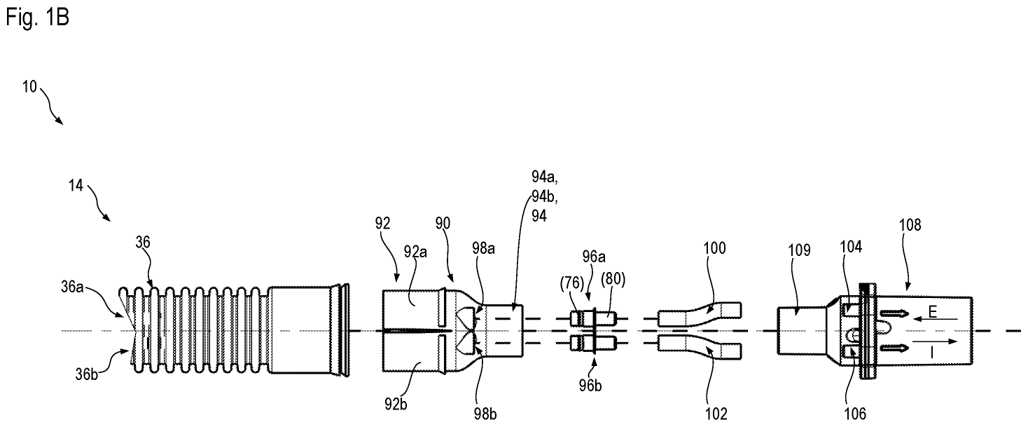

Referring now to the drawings wherein the showings are for the purpose of illustrating preferred and alternative embodiments of the invention only and not for the purpose of limiting the same, in FIGS. 1A to 3B, an embodiment of a breathing gas line assembly according to the invention is denoted generally by 10. FIGS. 1A, 2A, and 3A depict the distal longitudinal end region 12 of the breathing gas line assembly 10, FIGS. 1B, 2B, and 3B its proximal longitudinal end region 14.

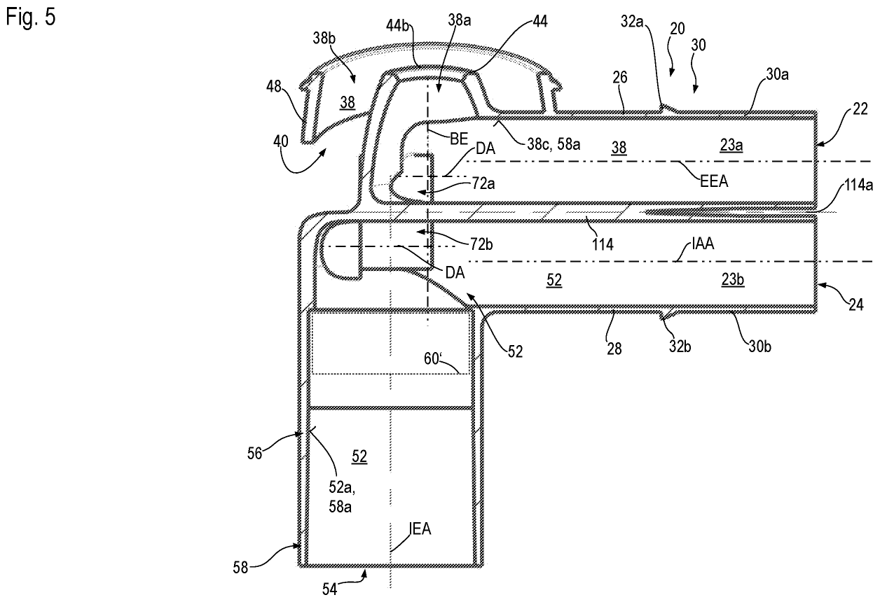

The distal longitudinal end 12 of the breathing gas line assembly 10, that is, the one located further away from the ventilated patient, comprises an expiration valve assembly 16 with a duct component 18 integrally injection-molded from a synthetic. The duct component 18 on its own is shown in longitudinal section in FIG. 5.

The duct component 18 comprises a multi-lumen end section 20, which in its upper region in FIG. 1A exhibits an expiration inlet 22 and in its lower region an inspiration outlet 24. To the expiration inlet 22 there links in the expiration direction E a straight expiration inlet section 26 which extends along an expiration inlet axis EEA. To the inspiration outlet 24 there links against the inspiration direction I a straight inspiration outlet section 28 which extends along an inspiration outlet axis IAA. The expiration inlet axis EEA and the inspiration outlet axis IAA are parallel to one another in the depicted embodiment example, likewise the expiration inlet section 26 and the inspiration outlet section 28.

The multi-lumen end section 20 exhibits a common linkage connector formation 30 which in its upper region in FIG. 1A acts as an inlet linkage formation 30a and in its lower region in FIG. 1A acts as an outlet connector formation 30b. Radial projections 32a and 32b, which act as end stops for a cooperating linkage connector counter-formation 34 of the multi-lumen hose as the multi-lumen line component 36 of the breathing gas line assembly 10, indicate the longitudinal end of the inlet linkage formation 30a and of the outlet connector formation 30b.

The multi-lumen hose 36 can be pushed with its linkage connector counter-formation 34 onto the linkage connector formation 30 of the multi-lumen end section 20, in order to establish a flow connection between the multi-lumen hose 36 and the duct component 18. The linkage connector counter-formation 34 can be held detachably, frictionally engaged or with positive fitting, at the linkage connector formation 30, for example through a bayonet catch or through a sprung latching or through other fastening means.

In the expiration valve assembly there is configured an expiration duct 38 which proceeds from the expiration inlet 22 to an expiration outlet 40. The expiration inlet section 26 is part of the expiration duct 38.

In the expiration duct 38 there is arranged in the expiration direction E between the expiration inlet 22 and the expiration outlet 40 an expiration valve 42 in the form of a membrane valve. In FIG. 1A there can be discerned the annular, planar, but tilted valve seat 44 at which in the expiration direction E an inlet-side expiration duct section 38a ends, and from which in the expiration direction E an outlet-side expiration duct section 38b originates. The expiration duct sections 38a and 38b are separated from one another by the expiration valve 42. The expiration valve 42 exhibits to this end a valve body 46 in the form of a membrane, which is held at the duct wall 48 of the outlet-side expiration duct section 38b by a lid component 50. The membrane valve body 46 can be clamped between the duct wall 48 and the lid component 50.

If expiratory breathing gas flows in the expiration direction E through the inlet-side expiration duct section 38a onto the membrane valve body 46, the latter is raised from the annular valve seat 44 through the building pressure against the pre-tensioning force exerted by the material elasticity of the membrane valve body 46, and the expiratory breathing gas can overflow from the inlet-side expiration duct section 38a into the outlet-side expiration duct 38b surrounding it radially outside.

In the expiration valve assembly 16, in particular in the integral duct component 18, there is configured an inspiration duct 52 which can be discerned most clearly in FIG. 5. It extends in the inspiration direction I from an inspiration inlet 54 to the inspiration outlet 24.

To the inspiration inlet 54 there links in the inspiration direction I a straight inspiration inlet section 56 as part of the inspiration duct 52, proceeding along an inspiration inlet axis IEA which in the depicted example encloses with the inspiration outlet axis IAA an angle of 90°. A region of the inspiration inlet section 56 exhibiting the inspiration inlet 54 is configured as an inlet connector formation 58 which serves to establish a connection conducting inspiratory breathing gas in the inspiration direction I, with which the inspiratory breathing gas can be introduced from a breathing gas source not depicted in the drawings, for instance from a ventilator, into the inspiration duct 52. The inlet connector formation 58 can be configured as a plug-in socket which can be plugged onto a linkage fitting at the breathing gas source, surrounding the latter radially outside. Alternatively, the inlet connector formation 58 can be configured as a linkage fitting which can be plugged into a socket at the breathing gas source.

Into the inspiration inlet section 56 there is introduced a one-way valve 60 which exhibits a basic component 62 and an elastomeric disc-shaped valve body 64 attached to it. The one-way valve 60 is welded with an inner wall 52a (see FIG. 5) of the inspiration duct 52, preferably through ultrasound welding of mounting brackets 62a of the basic component 62 with the inner wall 52a, and thus positionally secured. The valve body 64 is attached in a central region with positive fit to the basic component 62 and under an inflow in the inspiration direction I can be raised with its circumferential edge region from a valve seat 62b configured at the basic component 62. Under an inflow against the inspiration direction I, the valve body 64 is pressed against the valve seat 62b, thus closing the inspiration duct 52.

In analogy with the inspiration duct 52, the expiration duct 38 is bounded by a duct wall 38c. The duct wall 48 of the outlet-side expiration duct section 38b is part of the duct wall 38c.

A dotted rectangle 60′ indicates in FIG. 1 the position of the one-way valve 60 in the duct component 18 in the fully mounted state. In FIG. 1, the disc-shaped valve body 64 is oriented in such a way that the extension plane of the valve body disc lies orthogonally to the drawing plane of FIG. 1.

From the inspiration duct 52 there branches off from a branching point 66, which in the inspiration direction I lies upstream of the built-in valve body 64 of the one-way valve 60, a control duct 68 which by means of an aperture 70 in the membrane valve body 46 penetrates through the membrane valve body 46 and ends in a chamber of the lid component 50 located on the side facing away from the valve seat 44 of the expiration valve 42. Thus, regardless of the operational state of the one-way valve 60, inspiratory breathing gas can always be conducted through the control duct 68 on the side of the membrane valve body 46 facing away from the valve seat 44, such that during an inspiration process the expiration valve 42 can be held by the inspiration breathing gas securely in its blocking position, in which the membrane valve body 46 resting on the valve seat 44 blocks flow through the expiration duct 38. The control duct 68 lies section-wise structurally both outside the inspiration duct 52 and outside the expiration duct 38 and at least along one section has no wall section in common with the inspiration duct 52 or the expiration duct 38 in such a way that the common wall section would on one side bound the control duct 68 and on its opposite side a duct out of the inspiration duct 52 and expiration duct 38. Only in the region of the duct wall 48 of the outlet-side expiration duct section 38b there exists a wall section which bounds both a section of the control duct 68 and a section of the outlet-side expiration duct section 38b.

In order to be able to hold the expiration valve securely closed during an inspiration process, the inspiratory membrane area 46a of the membrane valve body 46 which is wettable by inspiratory breathing gas is larger than the opposite expiratory membrane area 46b which when the expiration valve 46 is closed lies inside the valve seat 44 and can have an inflow of expiratory breathing gas from the inlet-side expiration duct section 38a. In the depicted preferred case, the inspiratory membrane area 46a is 1.8 times larger than the expiratory membrane area 46b.

The duct component 58 exhibits flow-through apertures 72a in the expiration duct 38 and 72b in the inspiration duct 52 which are discernible in FIG. 5. The flow-through apertures 72a and 72b, each of which proceeds along a feed-through axis DA which is parallel to the expiration inlet axis EEA and to the inspiration outlet axis IAA, penetrate through the duct component wall 58a of the duct component 58 which bounds both the inspiration duct 52 and the expiration duct 38.

In the flow-through apertures 72a and 72b there are built-in feedthrough components 74a and 74b respectively, depicted in FIG. 1 and configured separately from the duct component 18, each extending along the assigned feed-through axis DA. Since the feedthrough components 74a and 74b are identical, there suffices the description of just the feedthrough component 74a which also applies to the other feedthrough component 74b.

The feedthrough component 74a exhibits on its side which faces into the expiration duct 38, i.e. into the inner region of the duct component 18, an accommodating formation 76 with which a first signaling line 78 led in the expiration lumen 36a of the multi-lumen hose 36 is positively connectable. The accommodating formation 76 can be a flange surrounding the signaling line 78, a spring ring, or a plug-in fitting onto which the signaling line 78 can be plugged.

On its opposite side the feed-through component 74a exhibits a further accommodating formation 80, with which a further signaling line 82 is positively connectable. The further accommodating formation 80 too, can be a flange, a spring ring, or a plug-in fitting.

In the multi-lumen hose 36, there is passed in the inspiration lumen 36b a second signaling line 84 which in analogy with the feedthrough component 74a described above can be continued, by means of the feedthrough component 74b, on the outside of the duct component 18 by a yet further signaling line 86.

The signaling lines 78 and 84 and the further signaling lines 82 and 86 transmit as hollow lines pressure information from a flow sensor 108 described further below in the proximal longitudinal end region 14 of the breathing gas line assembly 10. The further signaling lines 82 and 86 can lead into a plug 88, with which simply and securely a connection transmitting the pressure information can be established with a pressure sensor in a ventilator.

FIG. 1B shows the proximal longitudinal end 14 of the breathing gas line assembly 10, that is, the one which during operation lies nearer to the ventilated patient. The multi-lumen hose 36 is constructed at its distal longitudinal end identically with the proximal longitudinal end already described above, such that reference is made to the description of the proximal longitudinal end of the multi-lumen hose 36 for elucidating the distal longitudinal end.

To the proximal longitudinal end of the multi-lumen hose 36 there is coupled a proximal coupling component 90 configured as a separate component, which at its distal longitudinal end exhibits a common linkage connector formation 92 which corresponds to the linkage connector formation 30 already described above, to the description of which reference is made for elucidating the linkage connector formation 92 also. The common linkage connector formation 92 comprises an upper expiration connector formation 92a in FIG. 1B which corresponds to the inlet connector formation 30a at the duct component 18, and comprises a lower inspiration connector formation 92b in FIG. 1B which corresponds to the outlet connector formation 30b at the duct component 18. The proximal coupling component 90 is in its function a Y-connector component, since at its proximal longitudinal end it merges the expiration lumen and the inspiration lumen into a common coupling formation 94 which is both a proximal expiratory coupling formation 94a and a proximal inspiratory coupling formation 94b. Therefore, the common coupling formation 94 has both inspiratory and expiratory breathing gas flowing through it where the aforementioned breathing gases, depending on the positions of the expiration valve 42 and of the one-way valve 60, flow in the lumina assigned to them out of expiration lumen and inspiration lumen. The common coupling formation 94 is connectable, for example pluggable, with an input linkage formation 109 at the distal longitudinal end of a flow sensor 108.

The first signaling line 78 proceeding in the expiration lumen 36a and the second signaling line 84 proceeding in the inspiration lumen 36b are not shown in FIG. 1B. However, they are present and lead again into feedthrough components 96a and 96b, which are identical with the feedthrough components 74a and 74b described earlier. The feedthrough components 96a and 96b are each arranged in a flow-through aperture 98a and 98b respectively penetrating through the wall of the proximal coupling component 90. Outside the coupling component 90, the signaling lines 78 and 84 are continued through further proximal signaling lines 100 and 102 which end at a linkage fitting 104 and 106 respectively of a differential pressure flow sensor 108. The signaling lines 100 and 102, 80 and 84, as well as 82 and 86 transmit as hose lines the pressures on both sides of a flow resistance, not depicted in further detail but known per se, inside the differential pressure flow sensor 108 to a pressure sensor in the ventilator, which from the transmitted pressure data ascertains during the inspiration and the expiration the breathing gas flows passing bidirectionally through the proximal differential pressure flow sensor 108.

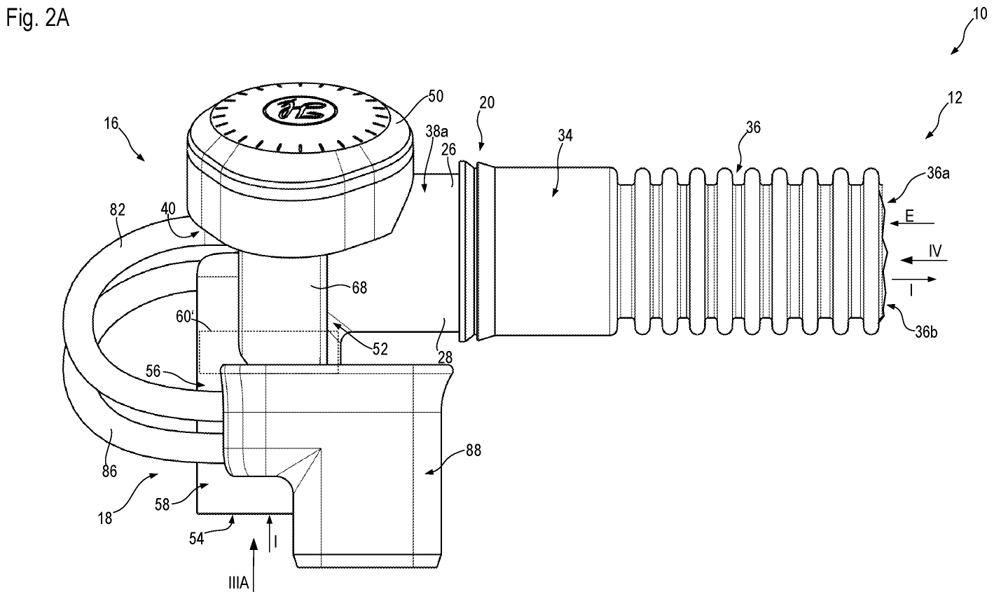

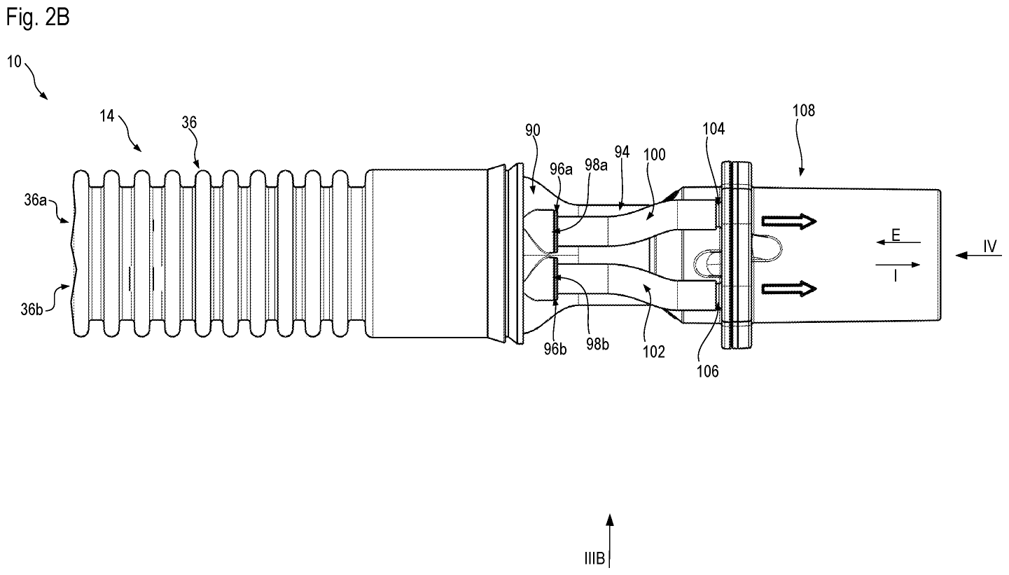

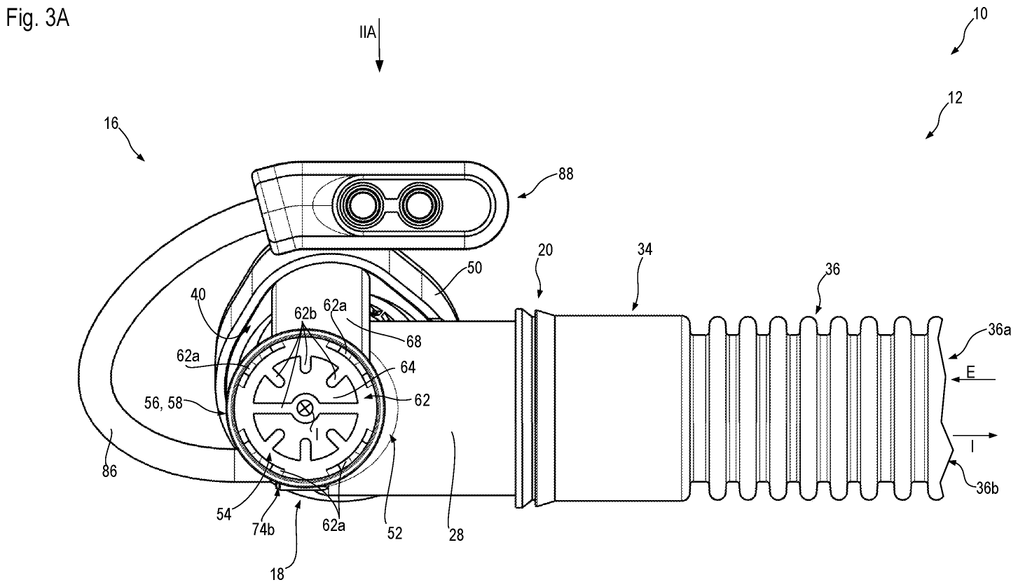

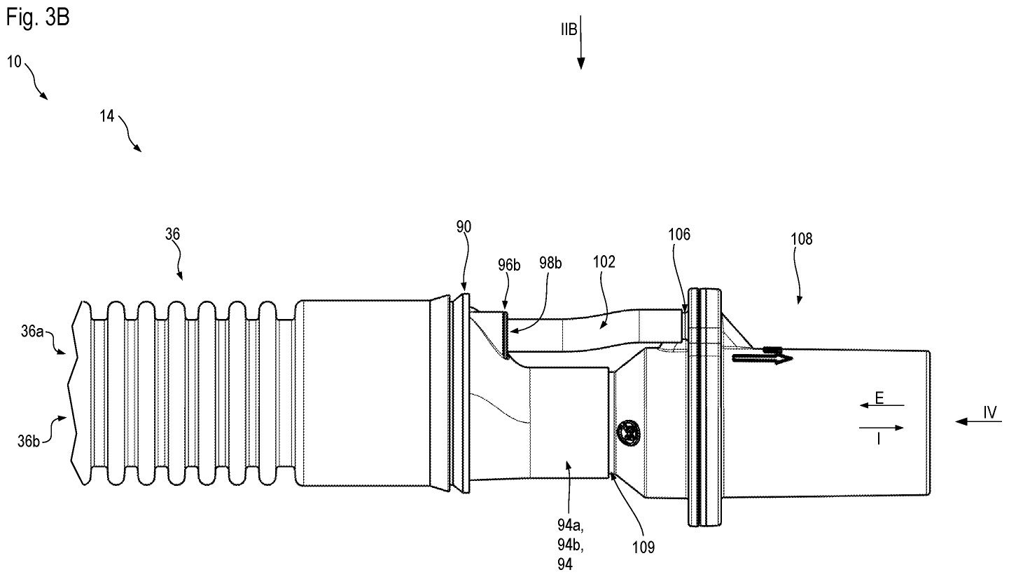

In FIGS. 2A and 3A there is depicted the distal longitudinal end of the breathing gas line assembly 10 in the installed state from the directions of view named in the above listing of figures and depicted in the drawings. The equivalent applies in FIGS. 2B and 3B to the proximal longitudinal end of the breathing gas line assembly 10.

In the bottom view of FIG. 3A there can be discerned the structure of the basic component 62 with the mounting brackets 62a and the valve seat 62b of the one-way valve 60, as well as the valve body 64 situated behind is when viewing FIG. 3A. For the sake of improved clarity, not all structures of the valve seat 62b are labelled with a reference mark.

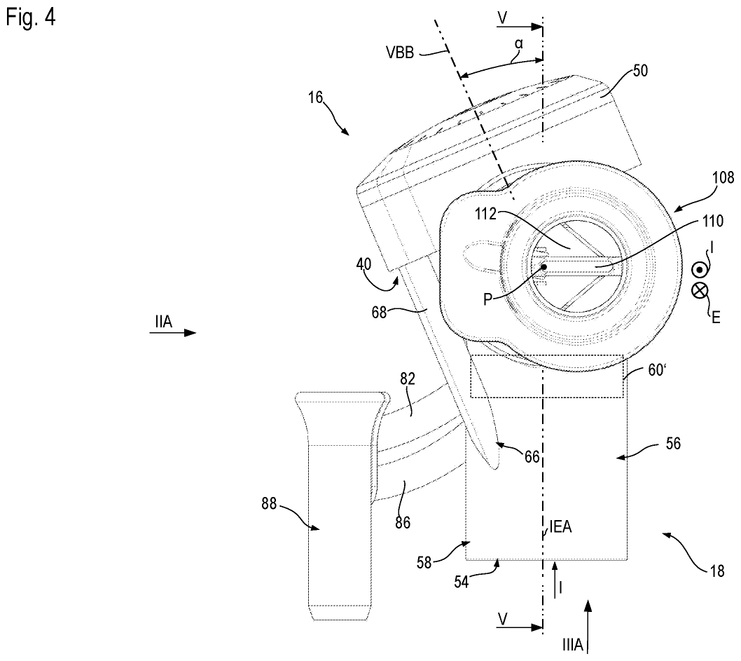

In FIG. 4, the breathing gas line assembly 10 is viewed from the proximal end of the differential pressure flow sensor 108 along a parallel P to the expiration inlet axis EEA and to the inspiration outlet axis IAA which is orthogonal to the drawing plane of FIG. 4. Aside from looking in FIG. 4 into the proximal aperture of the flow sensor 108 through which both inspiratory and expiratory breathing gas flow and there discerning a flow-guiding element 110 proceeding orthogonally to the drawing plane of FIG. 4 and a foil-like flow resistance component 112 with flow-dependent variable flow resistance lying behind it in parallel to the drawing plane of FIG. 4, FIG. 4 shows also the tilt of the lid component 50 with respect to the inspiration inlet axis IEA about the parallel line P. The tilt angle α equals in the depicted example between 20 and 30°, preferably between 22 and 26°, especially preferably 24° or 25°.

Through this construction, the valve movement path VBB along which the membrane valve body 46 rises from the valve seat 44 for a movement towards its feed-through position and moves towards the valve seat 44 to return to its blocking position, is also tilted by the tilt angle α. Thereby there is obtained an expiration valve assembly 16 in which even during the hectic activity at accident sites and in rescue situations the gravitational force contributes to straining of the membrane valve body 64 in the direction towards the valve seat 44, thus supporting a kind of pre-tensioning of the expiration valve 42 into the blocking position. This straining through the gravitational force adds to the pre-tensioning of the expiration valve 42 into the blocking position due to the construction and the material elasticity of the membrane valve body 64.

The control duct 68 preferably proceeds in parallel to the valve movement path VBB.

Through the tilting of the valve movement path VBB and thus of the lid component 50, the lid component 50, but first and foremost the expiration valve 42 and in particular the valve seat 44 which runs orthogonally to the valve movement path VBB, exhibits an proximity section 44a shown in FIG. 1 which lies nearer to the inspiration inlet 54 and a distanced section 44b which lies further away from the inspiration inlet 54. The distanced section 44b and the proximity section 44a lie diametrically opposite one another with respect to the valve movement path VBB. In order to be able to realize an especially short control duct 68, it is situated on the side of the proximity section 44a.

FIG. 5 shows a longitudinal section through the duct component 18 along the sectional plane V-V shown in FIG. 4. There too, the distanced section 44b of the valve seat 44 can be discerned.

FIG. 5 likewise shows the flow-through apertures 72a and 72b and the septum 114 which is oriented orthogonally to the drawing plane of FIG. 5 and is essentially planar, and which subdivides the multi-lumen end section 20 in the duct component 18 into an expiration lumen 23a and an inspiration lumen 23b. In the fully installed state of the breathing gas line assembly 10, the lumina expiration lumen 23a and inspiration lumen 23b continue in the multi-lumen hose 36 in the expiration lumen 36a and the inspiration lumen 36b respectively. The multi-lumen hose 36 too, is subdivided into the two lumina 36a and 36b through an essentially planar septum crossing it diametrically. A groove 114a configured in the septum 114 serves for the gas-tight connecting of the two septa of the multi-lumen end section 20 and of the multi-lumen hose 36.

In FIG. 5, the reference mark BE labels a reference plane BE orthogonal to the drawing plane of FIG. 5, which subdivides an aperture area surrounded by the valve seat 44 into equal area portions lying on the two sides of the reference plane BE. The feed-through apertures 72a and 72b lie in the expiration direction E downstream of the reference plane BE. The signaling lines 78 and 84 can thereby proceed as long as possible as little curved as possible.

While considerable emphasis has been placed on the preferred embodiments of the invention illustrated and described herein, it will be appreciated that other embodiments, and equivalences thereof, can be made and that many changes can be made in the preferred embodiments without departing from the principles of the invention. Furthermore, the embodiments described above can be combined to form yet other embodiments of the invention of this application. Accordingly, it is to be distinctly understood that the foregoing descriptive matter is to be interpreted merely as illustrative of the invention and not as a limitation.

1-15. (canceled)

16. An expiration valve assembly for a respiratory device for artificial ventilation of a patient, comprising
an expiration duct which at one end exhibits an expiration inlet for introducing an expiratory breathing gas flow into the expiration duct and an inlet linkage formation which is configured for linking to an expiration breathing gas line leading to the patient, and which at its other end exhibits an expiration outlet for discharging expiratory breathing gas, where the expiration duct exhibits an expiration valve which through an expiratory breathing gas flow in the expiration direction from the expiration inlet to the expiration outlet is moveable into a feed-through position which lets the expiratory breathing gas flow through,
an inspiration duct which exhibits at one end an inspiration inlet for introducing an inspiratory breathing gas flow into the inspiration duct and an inlet connector formation which is configured for connecting with a breathing gas source supplying inspiratory breathing gas, and which at its other end exhibits an inspiration outlet for letting out inspiratory breathing gas and an outlet connector formation which is configured for connecting with an inspiration breathing gas line leading to the patient,
a control duct which branches off from the inspiration duct at a branching point and leads to the expiration valve in such a way that the expiration valve can be strained by inspiratory breathing gas into a blocking position which blocks the expiratory breathing gas flow,
wherein in the inspiration duct there is arranged a one-way valve which allows inspiratory breathing gas flow in the inspiration direction from the inspiration inlet to the inspiration outlet and prevents breathing gas flow in the opposite direction.

17. The expiration valve assembly according to claim 16, wherein the branching point lies upstream of the one-way valve in the inspiration direction.

18. The expiration valve assembly according to claim 16, wherein an inspiration inlet section of the inspiration duct which lies nearer to the inspiration inlet than to the inspiration outlet proceeds along an inspiration inlet axis, where a valve movement path along which a valve body of the expiration valve can be raised from a valve seat of the expiration valve from its blocking position and made to approach the valve seat, is tilted relative to the inspiration inlet axis by a setting angle in the range from 10° to 45°.

19. The expiration valve assembly according to claim 18, wherein the setting angle is in the range from 15° to 35°.

20. The expiration valve assembly according to claim 18, wherein an expiration inlet section of the expiration duct which lies nearer to the expiration inlet than to the expiration outlet proceeds along an expiration inlet axis, where the valve movement path is tilted about a parallel to the expiration inlet axis with respect to the inspiration inlet axis.

21. The expiration valve assembly according to claim 18, wherein the valve seat exhibits a proximity section approaching and tilted towards the inspiration inlet section and a distanced section lying further away and tilted away from the inspiration inlet section, where the control duct runs nearer to the proximity section than to the distanced section from the branching point to the expiration valve.

22. The expiration valve assembly according to claim 20, wherein in a region between the inspiration inlet section and the expiration inlet section and/or in a region between the inspiration inlet section and an inspiration outlet section lying nearer to the inspiration outlet than to the inspiration inlet there is configured at least one feed-through aperture which penetrates through a duct wall which bounds the inspiration duct and/or the expiration duct.

23. The expiration valve assembly according to claim 22, wherein the at least one feed-through aperture is located in the expiration direction downstream of a reference plane which is oriented orthogonally to the expiration inlet axis and subdivides an aperture surface bordered by the valve seat into equal area parts.

24. The expiration valve assembly according to claim 22, wherein the at least one feed-through aperture runs along a feed-through axis which is parallel to the expiration inlet axis and/or to an inspiration outlet axis along which the inspiration outlet section extends.

25. The expiration valve assembly according to claim 16, wherein an expiration inlet section of the expiration duct which lies nearer to the expiration inlet than to the expiration outlet proceeds along an expiration inlet axis, where the valve movement path is tilted about a parallel to the expiration inlet axis with respect to the inspiration inlet axis; in a region between the inspiration inlet section and the expiration inlet section and/or in a region between the inspiration inlet section and an inspiration outlet section lying nearer to the inspiration outlet than to the inspiration inlet there is configured at least one feed-through aperture which penetrates through a duct wall which bounds the inspiration duct and/or the expiration duct; the expiration inlet section and the inspiration outlet section are configured in a common multi-lumen end section.

26. The expiration valve assembly according to claim 16, wherein an expiration inlet section of the expiration duct which lies nearer to the expiration inlet than to the expiration outlet proceeds along an expiration inlet axis, where the valve movement path is tilted about a parallel to the expiration inlet axis with respect to the inspiration inlet axis; in a region between the inspiration inlet section and the expiration inlet section and/or in a region between the inspiration inlet section and an inspiration outlet section lying nearer to the inspiration outlet than to the inspiration inlet there is configured at least one feed-through aperture which penetrates through a duct wall which bounds the inspiration duct and/or the expiration duct; the inspiration inlet section, the expiration inlet section, and the inspiration outlet section are realized through an integral duct component.

27. A breathing gas line assembly, comprising an expiration valve assembly according to claim 16, an expiration breathing gas line, an inspiration breathing gas line, and a flow sensor,
where the expiration breathing gas line exhibits at its distal longitudinal end a distal expiratory coupling formation which is configured to establish a connection which conducts an expiratory breathing gas flow with the inlet linkage formation of the expiration duct of the expiration valve assembly,
where the expiration breathing gas line exhibits at its proximal longitudinal end a proximal expiratory coupling formation which is configured to establish a connection which conducts an expiratory breathing gas flow with an expiratory output linkage formation at the distal longitudinal end of the flow sensor,
where the inspiration breathing gas line exhibits at its distal longitudinal end a distal inspiratory coupling formation which is configured to establish a connection which conducts an inspiratory breathing gas flow with the outlet connector formation of the inspiration duct of the expiration valve assembly,
where the inspiration breathing gas line exhibits at its proximal longitudinal end a proximal inspiratory coupling formation which is configured to establish a connection which conducts an inspiratory breathing gas flow with an inspiratory input linkage formation at the distal longitudinal end of the flow sensor,
where in at least one breathing gas line out of the expiration and inspiration breathing gas line there is accommodated at least one signaling line which is configured to transmit information acquired by the flow sensor from the proximal to the distal longitudinal end of the at least one breathing gas line.

28. The breathing gas line assembly according to claim 27, wherein in a region between the inspiration inlet section and the expiration inlet section and/or in a region between the inspiration inlet section and an inspiration outlet section lying nearer to the inspiration outlet than to the inspiration inlet there is configured at least one feed-through aperture which penetrates through a duct wall which bounds the inspiration duct and/or the expiration duct; at least one of the at least one signaling line penetrates through at least one of the at least one feed-through aperture or ends in an accommodating engagement of an accommodating formation for accommodating the distal end of the at least one signaling line.

29. The breathing gas line assembly according to claim 27, wherein the expiration breathing gas line and the inspiration breathing gas line are configured at a common multi-lumen line component.

30. The breathing gas line assembly according to claim 29, wherein in the expiration lumen and in the inspiration lumen there is a signaling line accommodated in each.

31. The breathing gas line assembly according claim 27, wherein the breathing gas line assembly exhibits a proximal coupling component with which the multi-lumen line component is connected, where the coupling component exhibits at its distal longitudinal end an expiration connector formation for establishing a connection which conducts an expiratory breathing gas flow with the expiration lumen and an inspiration connector formation for establishing a connection which conducts an inspiratory breathing gas flow with the inspiration lumen, and where the coupling component exhibits at its proximal longitudinal end a coupling formation which is both the proximal expiratory coupling formation and the proximal inspiratory coupling formation.
